(19) United States
(12) Patent Application Publication
(10) Pub. No.: US 2026/0155064 A1
(43) Pub. Date: Jun. 4, 2026

(54) SURGICAL CONFIGURATION MODEL

(71) Applicant: NEXUS SPINE, LLC, Salt Lake City, UT (US)

(72) Inventors: David T. Hawkes, Salt Lake City, UT (US); Peter A. Halverson, Salt Lake City, UT (US)

(21) Appl. No.: 19/405,113

(22) Filed: Dec. 1, 2025

Related U.S. Application Data
(60) Provisional application No. 63/727,104, filed on Dec. 2, 2024.

(51) Int. Cl. G09B 23/32; G01C 11/00; G09B 23/34

(52) U.S. Cl. CPC G09B 23/32; G01C 11/00; G09B 23/34

(57) ABSTRACT

A spinal configuration model, and systems for implementing the same, are disclosed. In some implementations, the spinal configuration model includes one or more proxies configured to simulate the position of one or more surgical components during a surgical procedure. Accordingly, one or more implant components can be formed or modified based on simulated positions, thus simplifying the implant formation and installation process. Other implementations are also described.

### CROSS-REFERENCE TO RELATED APPLICATION AND PRIORITY CLAIM

This application claims priority to U.S. Provisional Patent Application Ser. No. 63/727,104, filed Dec. 2, 2024, and entitled SURGICAL CONFIGURATION MODEL (Attorney Docket No. 23845.203); the entire disclosure of which is incorporated herein by reference.

### FIELD

The present disclosure relates to medical devices, and more particularly to tools for assisting in the performance of surgery.

### BACKGROUND

Surgery often involves alignment of components, insertion of implants, making modifications to the human body, or other complex steps that must be performed with high precision and accuracy to ensure favorable outcomes. Unfortunately, it can sometimes be extremely difficult to visualize the inner workings of the human body during surgery. Oftentimes, one or more portions of a patient's body (e.g., skin, muscles, fascia, bone, or other body parts) obscure a surgeon's view of a target site, a surgical implant, or another important item during surgery. Accordingly, this can greatly increase the difficulty of performing surgeries correctly.

Thus, while techniques currently exist that are used to perform surgery, challenges can still exist, including those listed above. Accordingly, it would be an improvement in the art to augment or even replace current techniques with other techniques.

### SUMMARY

Various implementations of systems and methods for assisting in one or more aspects of a surgical procedure are provided. In some implementations, a surgical configuration model is provided. The surgical configuration model can include any component configured to simulate one or more aspects of a surgical procedure in an analog format (although one or more digital simulations may also be provided). For example, some iterations include a first proxy configured to represent a first surgical component. Some iterations include a second proxy configured to represent a second surgical component. Additional iterations can include any additional number of proxies configured to represent third, fourth, fifth, sixth, or any other number of additional surgical components.

Some implementations include one or more surgical configuration mechanisms configured to cause the proxies to simulate one or more positions of the surgical components that they represent (e.g., during a surgical procedure). For example, where a first anchor and a second anchor (such as pedicle screws in a patient's spine) are some of the surgical components in question, the first proxy can represent the first anchor and the second proxy can represent the second anchor (and the proxies can assume the positions of the respective anchors). Then a practitioner can use or evaluate the proxies instead of experimenting on the actual anchors in the patient's back.

In some cases, one or more actuation assemblies are included. For example, in some cases, the first proxy is coupled to a first actuator configured to adjust a position of the first proxy. In some cases, the second proxy is also coupled to the first actuator, which is additionally configured to adjust the position of the second proxy. That said, some implementations of the second proxy are coupled to a second actuator configured to adjust the position of the second proxy. Thus, any suitable number of actuators can be used. Additionally, in some cases, the actuators (or any other suitable portion or portions of the described systems) are configured to collect data regarding the proxies (e.g., position, displacement, or other information).

In some cases, one or more proxies (e.g., the first proxy or any other proxy) are selectively interchangeable. In some cases, one or more proxies include a proxy head that is selectively interchangeable with any other suitable proxy head.

In some implementations, the surgical configuration model includes a housing. In some cases, the first proxy is configured to extend from an opening in (or other portion of) the housing. In some cases, a lid for covering the opening (or a portion of the housing) is included. Thus, some implementations allow for protection of the interior components of the surgical configuration model (e.g., the surgical configuration mechanism) by encasing them in the housing.

According to some implementations, a system is provided. Some iterations of the system include the surgical configuration model (or any part thereof). Some iterations of the system include one or more sensors configured to sense (e.g., measure, calculate, identify, monitor, or otherwise ascertain) a position of the first component (or any other component) during the surgical procedure. In some cases, the spinal configuration model is configured (e.g., manually, automatically, or in any other suitable manner) to mimic the positions of components as measured.

In some embodiments, the system includes or is otherwise used with software configured to communicate with (e.g., in communication with) the surgical configuration mechanism. In some cases, the software is configured to calculate a desirable position of the first component and to cause the surgical configuration mechanism to place the first proxy in a position simulating the desirable position of the first component. In some cases, the same applies to a second component and a second proxy, or any additional number of components and proxies.

In some cases, the system (e.g., the sensor, or any other suitable part of the system) includes a stereotactic camera or sensor array (such as one having a first camera and a second camera) for more accurate position measurement.

Some embodiments include one or more methods for modeling one or more aspects of a surgical procedure. The method can include use of any component as discussed herein, but in some cases the method includes obtaining a surgical configuration model (e.g., any surgical configuration model as set forth above or otherwise described herein). In some cases, the method includes causing the first proxy to simulate the position of the first surgical component. In some cases (where a second proxy is included), the method includes causing the second proxy to simulate a position of the second surgical component with respect to the position of the first surgical component.

In some cases, causing the first proxy to simulate the position of the first surgical component includes causing the first proxy to simulate at least one of an x-location, a y-location, a z-location, and an orientation of the first proxy.

In some implementations, the method further includes obtaining a stereotactic localization system, using the stereotactic localization system to detect the position of the first surgical component, and using software to cause the surgical configuration mechanism to adjust the first proxy in accordance with data obtained from the stereotactic localization system.

In some implementations, the surgical configuration mechanism is configured to detect a change in the position of the first proxy, and the method further includes manually adjusting the position of the first proxy. In some cases, the method further includes detecting the change in the position of the first proxy using the surgical configuration mechanism.

Some implementations of the method include configuring a second surgical component using the first proxy as a guide and installing the second surgical component proximate the first surgical component.

The systems and methods described herein have many other features and implementations, so a more detailed description is provided below.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1 shows a front perspective view of a surgical configuration model in accordance with some representative embodiments of the disclosed systems and methods;

FIG. 2 shows a top perspective view of a portion of a surgical configuration model, in accordance with some representative embodiments;

FIG. 3 shows a front perspective view of a mechanism from an interior of a surgical configuration model, in accordance with some representative embodiments;

FIG. 4 shows a plan view of a mechanism of a surgical configuration model, in accordance with some representative embodiments;

FIG. 5 shows a front perspective view of a mechanism of a surgical configuration model, in accordance with some representative embodiments;

FIG. 6 shows a front perspective view of a surgical configuration model, highlighting some features, in accordance with some representative embodiments;

FIGS. 7A and 7B depict X-ray images of a patient's spine with implants therein for illustrative purposes relating to the features of the surgical configuration model of FIG. 6;

FIGS. 8A and 8B show perspective views of multiple surgical configuration models used together, in accordance with some representative embodiments;

FIGS. 9A-9C show various proxy components for use in connection with surgical configuration models, in accordance with some representative embodiments;

FIG. 10 shows a perspective view into an interior of a surgical configuration model, in accordance with some representative embodiments;

FIGS. 11A-11E illustrate a procedure using an implant in connection with a patient's spine, as may be aided by a surgical configuration model, in accordance with some representative embodiments;

FIGS. 12A-12B illustrate a procedure using an implant in connection with a patient's spine, as may be aided by a surgical configuration model, in accordance with some representative embodiments;

FIGS. 13A-13C illustrate a procedure using an implant in connection with a patient's spine, as may be aided by a surgical configuration model, in accordance with some representative embodiments;

FIGS. 14A-14B illustrate a procedure using an implant in connection with a patient's mouth, as may be aided by a surgical configuration model, in accordance with some representative embodiments; and

FIGS. 15A-15D illustrate use of a surgical configuration model, in accordance with some representative embodiments.

### DETAILED DESCRIPTION

A description of embodiments will now be given with reference to the figures. It is expected that the present systems and methods may take many other forms and shapes. Hence, the following disclosure is intended to be illustrative and not limiting, and the scope of the disclosure should be determined by reference to the appended claims.

According to many surgical procedures and other operations as presently performed, it can be difficult for a practitioner to properly align or position various components during an operation. For example, where a surgeon needs to configure an implant to align with a number of surgical anchors that have already been inserted into a patient, it can be difficult for the surgeon to perform the alignment inside the patient's body, creating a need to make larger incisions or otherwise increase the difficulty, time, or cost of the procedure. Moreover, misalignment between components (e.g., anchors and a fixator configured to mate with such anchors) can result in damage to a patient's bones or other anatomical structures and can cause the patient pain. Furthermore, it can also be difficult to predict the outcome of attaching a fixator to one or more anchors. To illustrate, spinal alignment in scoliosis surgery is often not highly predictable. Indeed, many current operations require substantial operative time for shaping of implants during the operation (in some cases, requiring repeated insertion, measurement, removal, and reshaping before the final installation). Thus, spinal rod and other implant insertion could be greatly expedited by an ability to pre-form or pre-configure rods or other components prior to insertion.

The instant systems and methods are configured to assist practitioners in performing a variety of operations, such as surgical procedures. While the systems and methods described in this disclosure may do so in various ways, some embodiments include one or more surgical configuration models 20 configured to do one or more of the following: (a) simulate the actual position of one or more components (with respect to one or more body parts or one or more additional components), such as surgical components during surgery (e.g., implant components, such as anchors, rods, implant bodies, couplers, plates, fasteners, or any other implant components; instrument components, such as a handle, a shaft, a tip, a tool, or another portion of a surgical instrument; body parts of a patient, such as bones, muscles, tissues, spinal vertebrae, teeth, or any other body parts; or other components that would be useful to positionally track or have a positional model of during a procedure); (b) simulate a desirable position of one or more components (e.g., by adopting an ideal or desired configuration according to one or more calculations or as desired); (c) simulate (in an analog or a digital format) a different theoretical configuration of one or more components; and (d) create an analog or a digital representation of a position of one or more components. As examples, some embodiments include one or more housings 22 (which may have one or more openings) that are configured to house one or more proxies 26, surgical configuration mechanisms 28, actuation assemblies 34, or any other suitable components for performing any of the described functions.

The described surgical configuration model 20 can include any component in any configuration configured to simulate (in analog format, although some embodiments may additionally include one or more digital simulations) the actual, calculated, desirable, or theoretical positions of one or more surgical components. In this regard, some embodiments include one or more housings 22. The housing can include any suitable housing for containing the various parts of the surgical configuration model. It can be integrated into a fixed surface, such as a counter, tabletop, cart, or surgical space; it can comprise a stand-alone unit; it can be portable; or it can comprise any other suitable configuration. Where the surgical configuration model is portable, some embodiments are self-contained, relatively lightweight (e.g., less than 20 kg), or relatively small (e.g., less than 1m×1m×1m).

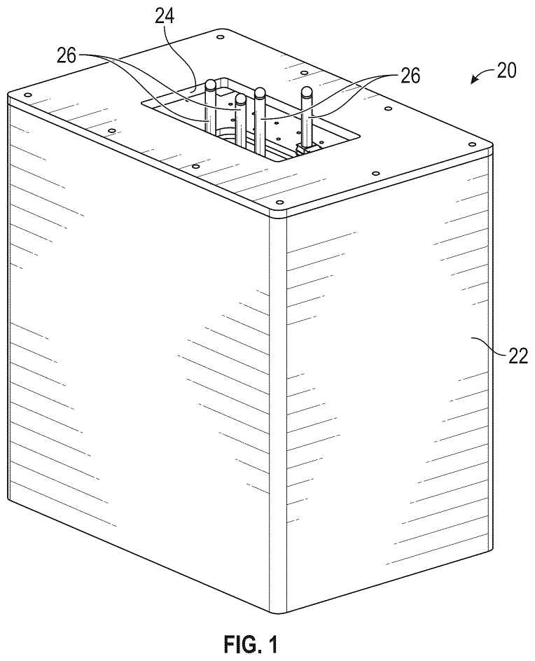

The housing 22 can be any shape (e.g., cuboidal, cylindrical, spherical, pyramidal, conical, prismatic, toroidal, polyhedral, tablet-shaped, or otherwise shaped). By way of non-limiting illustration, FIG. 1 shows some embodiments in which the housing 22 has a cuboidal shape.

Although any suitable portion of the housing 22 can have any suitable flexibility or rigidity, some embodiments are substantially rigid so as to provide protection for interior components. By way of non-limiting illustration, FIG. 1 shows a surgical configuration model 20 having a substantially rigid housing 22.

In some embodiments, the housing 22 includes one or more doors, windows, access points, or other openings 24 allowing visibility or access to one or more interior elements of the surgical configuration model, such as to one or more proxies 26 for simulating component positions. In some cases, one or more components (e.g., the proxies) protrude from or extend past a portion of the opening (or edge of the housing), whereas in some embodiments, most (if not all) components are entirely contained within the housing (e.g., they do not extend out of the opening or past an exterior edge or surface of the housing). In some embodiments, one or more components is configured to selectively extend out of the opening or beyond an edge of the housing (e.g., the housing is capable of containing all components within it, but the proxies or one or more other components can be actuated to extend out of the opening, beyond an edge or surface of the housing, or otherwise be transitioned from a stowed configuration (in which they are disposed entirely or mostly within the housing) to an active configuration (in which they extend from the opening). By way of non-limiting illustration, FIG. 1 shows a surgical configuration model 20 having a housing 22 with an opening 24 through which a number of proxies 26 extend.

In some embodiments, the opening 24 includes a lid or is otherwise configured to close to protect or better contain one or more of the interior components. Where a lid is included, the lid can include any suitable type of lid, including one or more hinged lids, sliding lids, snap-on lids, flexible covers, flexible shrouds, coverings, or any other suitable type of lid. The lid can be configured to cover just the opening, or it can be configured to cover an entire top (or other applicable portion) of the surgical configuration model 20.

As another example of a suitable opening 24, some embodiments include one or more access openings configured to allow access to the interior mechanisms of the surgical configuration model 20. For example, some embodiments include one or more access panels formed in the bottom, a side, or another suitable portion of the housing 22. In some cases, one or more surfaces is configured to be removed (e.g., to create an opening and allow for expanded access), such as the top, the bottom, a side, or any other suitable portion of the housing.

As mentioned above, some embodiments of the surgical configuration model 20 include one or more proxies 26. The proxies can include any suitable component configured to mimic, represent, or otherwise simulate a position of a surgical component, tool, portion of a patient (e.g., with respect to any other surgical component or portion of the patient), or any other suitable object associated with a surgical procedure. In this regard, the term “position” can include a location (e.g., an x-location, a y-location, a z-location), an orientation (e.g., an x rotation, a y-rotation, a z-rotation), a size, a shape, or any other positional characteristic (in general, or with respect to any other component). In some cases, the position can be broken into one or more constituent components, such as an x-position (including an x-location and an x-rotation), a y-position (including a y-position and a y-rotation), and a z-position (including a z-location and a z-rotation). Thus, a proxy can include any suitable component configured to simulate any positional characteristic (alone or in combination with any other positional characteristic). In some embodiments, the position of one or more proxies is fixed (e.g., for use as a reference point, a starting point, or an origin point), such as by being held stationary or being coupled to the housing 22 or another stationary component (in some cases, interchangeably or selectively).

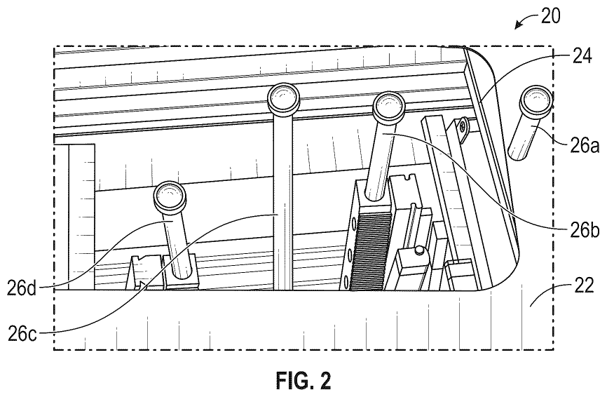

In some embodiments, the position of one or more proxies 26 is selectively adjustable (by a user or by the surgical configuration model itself). Furthermore, any number of proxies can be used (1, 2, 3, 4, 5, 6, 7, 8, 9, 10, or more). By way of non-limiting illustration, FIG. 2 shows a surgical configuration model 20 having a housing 22 with an opening 24 and a number of proxies 26, with a first proxy 26a being fixed to the housing 22, and with a second proxy 26b, a third proxy 26c, and a fourth proxy 26d extending through the opening 24 and being coupled to an interior surgical configuration mechanism (not clearly shown in FIG. 2) to allow for one or more of the proxies 26 to be selectively adjustable. Notwithstanding the foregoing, in some embodiments, all of the proxies are selectively adjustable (e.g., as opposed to being fixed, a reference proxy is also adjustable such that the position of the reference point can be set).

The proxies 26 can have any suitable shape. Indeed, the proxies can include any component configured to mimic, represent, or otherwise simulate any surgical component (e.g., a bone, a tool, an anchor, or any other component or portion of a patient). Some embodiments are configured to simulate specific components used in specific surgeries. For example, some proxies are configured to resemble or approximate one or more anchors (e.g., pedicle screws), rods, couplers, implant bodies, implants, wires, plates, vertebrae, sensors, markers, surgical instruments, or other surgical components. Indeed, some embodiments of the proxies include one or more proxy heads 27 configured to resemble or represent a specific component or portion thereof.

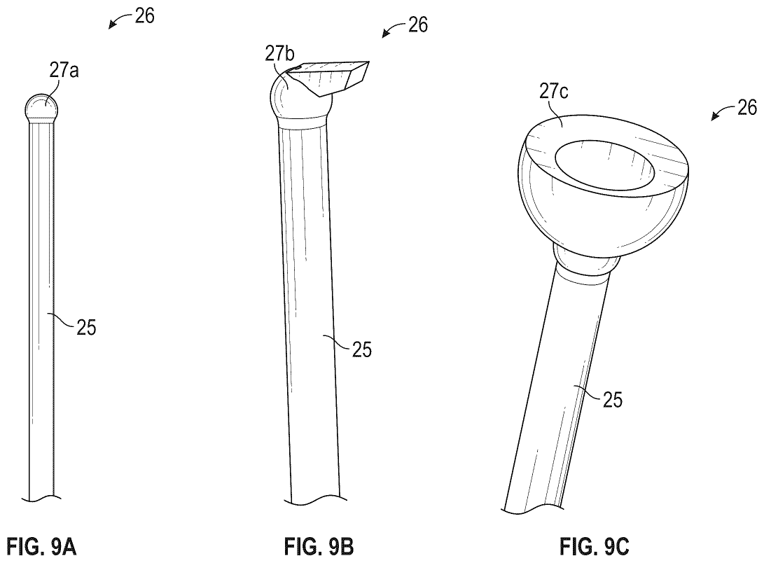

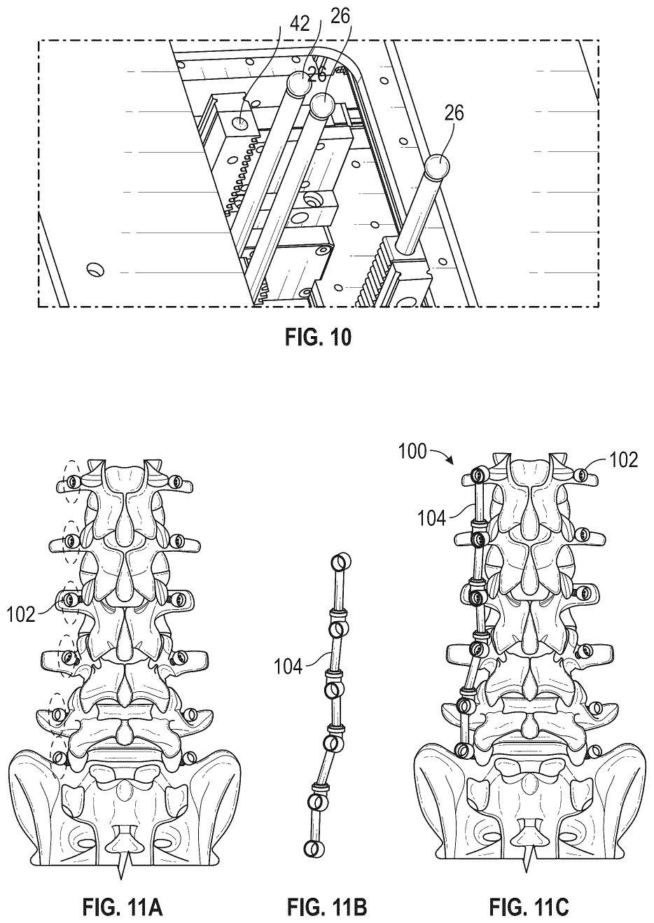

In some embodiments, one or more proxies 26 include one or more variable (e.g., adjustable or interchangeable) proxy heads 27. In some cases, the proxy heads are integral to the proxies (e.g., the whole proxy must be changed out for a different type of proxy to be used), but in some cases, the proxy heads themselves are selectively removable or interchangeable. For example, in some embodiments, one or more proxy heads are configured to be selectively attached and detached to a proxy to cause the proxy to adopt a desired configuration or characteristic for the surgical procedure in question. While the proxy heads can have any suitable configuration (e.g., being spherical, polygonal, cylindrical, conical, threaded, flanged, concave, convex, or any other suitable shape; being textured; comprising one or more magnets, mechanical engagements, frictional engagements, catches, fasteners, or any other suitable coupling mechanisms; or comprising any other suitable components or characteristics), some non-limiting examples of proxy heads 27 are shown in FIGS. 9A-9B. In this regard, FIG. 9A shows some embodiments of a proxy 26 having a pedicle screw configuration (e.g., having a pedicle screw proxy head 27a designed to provide at least 30 degrees of polyaxiality, similar to a screw). FIG. 9B shows some embodiments of a proxy 26 having a pedicle process configuration (e.g., having a pedicle process proxy head 27b configured to be used with or mimic one or more hooks or other pedicle processes). Moreover, FIG. 9C shows some embodiments of a proxy 26 having a socket configuration (e.g., having a socket proxy head 27c configured to mimic a socket into which a ball or other complementary component can be inserted). Thus, various proxies can be used to simulate anatomy, hardware, tool, or implants, and various proxies (or proxy heads) can be designed to simulate or account for implant (or other surgical component) capabilities and limits. In some embodiments, one or more kits are provided, with the kit including one or more proxies (including any suitable kind, such as the types discussed herein, in any suitable number and size (e.g., multiple sizes of a certain type are included in the kit, in some cases), and having any other characteristics as may be suitable). Where proxies 26 or proxy heads 27 are interchangeable, the surgical configuration model 20 can include any suitable feature for allowing such interchangeability. For example, some embodiments include one or more attachment points 42 (e.g., as shown in FIG. 10), at which one or more proxies (or proxy heads) can be selectively attached or detached. In some cases, one or more attachment points 42 are formed on or coupled (directly or indirectly) to one or more actuation assemblies 34 (as discussed in more detail below). Attachment points can include any suitable component for coupling to proxies, such as one or more sockets, threaded connections, bolts, eyelets, magnets, adhesives, welds, interference fits, friction fits, tongue-and-groove connections, mechanical engagements, detents, catches, rails, processes, recesses, snaps, ties, rivets, or any other suitable attachment mechanisms. By way of non-limiting illustration, FIG. 10 shows an attachment point 42 comprising a recess into which a process of a proxy 26 can be inserted.

The proxies 26 can be adjusted or moved in any suitable manner (e.g., manually; via one or more automated mechanisms, mechanical mechanisms, or selectively releasable locking mechanisms; or in any other manner or combination of manners). Indeed, in some embodiments, one or more proxies can be moved (e.g., manually or otherwise) and then be selectively locked in place. According to some embodiments, the proxies 26 are configured to be adjusted via a surgical configuration mechanism 28, as discussed in more detail below.

The proxies 26 can be coupled to such a surgical configuration mechanism 28 in any suitable manner. For example, some embodiments are coupled via one or more rods, cables, lattices, couplers, or any other coupling mechanism. That said, some embodiments include one or more shafts 25. Where a shaft is included, the shaft can include any suitable rod, bar, dowel, elongated component, or other shaft configured to couple the proxy head 27 to the surgical configuration mechanism. Some embodiments of the shaft have a sufficient length to extend from a portion of the interior of the surgical configuration model 20 (e.g., a lower tier 30b, as described in more detail below) through the opening 24 or beyond an external surface of the housing 22. By way of non-limiting illustration, FIGS. 9A-9C show proxies 26 having shafts 25 coupled to proxy heads 27a, 27b, 27c.

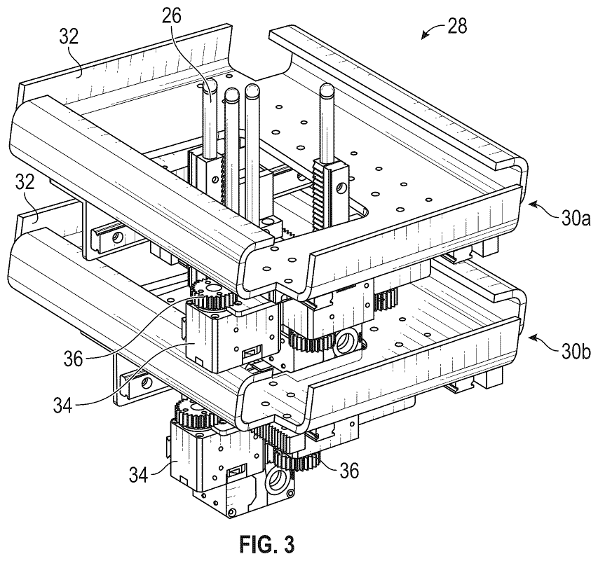

According to some embodiments, one or more of the proxies 26 are configured to be selectively adjustable via a surgical configuration mechanism 28. The surgical configuration mechanism can include any component configured to do any of the following: (a) automatically or selectively adjust the position of the proxy (e.g., to a calculated position, a position specified by a user, or another position); (b) allow for adjustment of a position of the proxy (e.g., by allowing a user to move a proxy to another position); (c) maintain the position of the proxy (e.g., by resisting or preventing movement of the proxy once it is in a desired position); or (d) sense a position of the proxy (e.g., to allow for accurate position, to allow for a digital representation of the position to be created, or for other reasons). While the surgical configuration mechanism have any suitable configuration and can function in any suitable manner, according to some embodiments, the surgical configuration mechanism 28 includes one or more tiers 30. Where it includes multiple tiers, a first tier 30a can control (e.g., be responsible for adjusting or maintaining the positions of) one or more proxies 26 in a first group (e.g., a first proxy and a second proxy, or a single proxy, or any additional number of proxies as may be in the first group), and a second tier 30b can control one or more proxies in a second group (e.g., a third proxy and a fourth proxy, or an additional single proxy, or any additional number of additional proxies as may be in the second group). Any additional number of tiers can also be included. In some embodiments with multiple tiers, the tiers are stacked vertically, and in some embodiments with multiple tiers, the tiers are stacked horizontally or in any other configuration. Some embodiments of the tiers are aligned along one or more axes (e.g., in some cases, a first edge of a first tier is directly above a corresponding first edge of a second tier, and so on for other edges or additional portions), whereas some embodiments of the tiers are offset along one or more axes. By way of non-limiting illustration, FIG. 3 shows some embodiments of a surgical configuration mechanism 28 with a first tier 30a stacked vertically on a second tier 30b, with each tier including additional components (discussed in more detail below) relating to one or more proxies 26, the tiers being substantially aligned along x-and y-axes while being offset (stacked) along a z-axis.

In some embodiments, the surgical configuration mechanism 28 includes one or more frames 32, which can have any shape or configuration for providing structure or support to the surgical configuration mechanism. For example, some embodiments of the frames are generally cuboidal, cylindrical, prismatic, polyhedral, polygonal, asymmetrical, symmetrical, or otherwise shaped in a manner to provide a scaffold, shielding, or other structure for additional components of the surgical configuration mechanism. By way of non-limiting illustration, FIG. 3 shows some embodiments of a surgical configuration mechanism 28 with a first tier 30a and a second tier 30b, each having a frame 32 with a main component that is substantially configured as an open-faced cuboid.

Although the frames 32 can be substantially 2-dimensional (e.g., formed of one or more generally flat materials), some embodiments of the frames have a 3-dimensional shape or contour. For example, some embodiments of the frame include a substantially sheet-like material with edges that are bent or that otherwise extend upward (or in an otherwise non-co-planar direction) to give the frame volume. In some embodiments, one or more additional components (e.g., one or more actuation assemblies 34, as described in additional detail below) is at least partially housed within the volume. In some embodiments, one or more parts of the frame (e.g., bent edges or any other suitable portion) separates one or more components from one or more other components (for example, they can separate one frame from another, or separate a first actuation assembly from a frame or from a second actuation assembly).

In some embodiments, one or more additional components of the surgical configuration mechanism 28 are mounted on or anchored to the frame 32 (e.g., the actuation assembly 34, or one or more of its components). That said, according to some embodiments, the frame includes one or more passages 33 through which one or more additional components are configured to extend (e.g., one or more proxies 26 or any other suitable components). By way of non-limiting illustration, FIG. 3 shows a surgical configuration mechanism 28 having two tiers 30a, 30b, with each tier having a frame 32 with a passage 33 formed therein, the passage 33 allowing passage of proxies 26 (and additional portions of the surgical configuration mechanism) therethrough.

As mentioned above, according to some embodiments, the surgical configuration mechanism 28 includes one or more actuation assemblies 34. The actuation assembly can include any component configured for positional adjustment or maintenance of one or more proxies 26, such as one or more actuators 36 for adjusting a proxy. The actuator can include any suitable type of actuator, including one or more motors (stepper motors, servomotors, bushed or brushless motors, induction motors, or any other motors), hydraulic actuators, pneumatic actuators, electric actuators, linear actuators (e.g., using rack-and-pinion mechanisms, belts, direct drive mechanisms, linear servo motors, or any other linear actuation mechanisms), piezoelectric actuators, thermal actuators, electromagnetic actuators, belt drives, geared mechanisms, dial controlled mechanisms, controller operated mechanisms, cable actuators, screw drives, micro-gear actuators, shape memory alloy actuators, fluidic actuators, magnetic actuators, mechanical actuators, hybrid actuation systems, or any other suitable actuators.

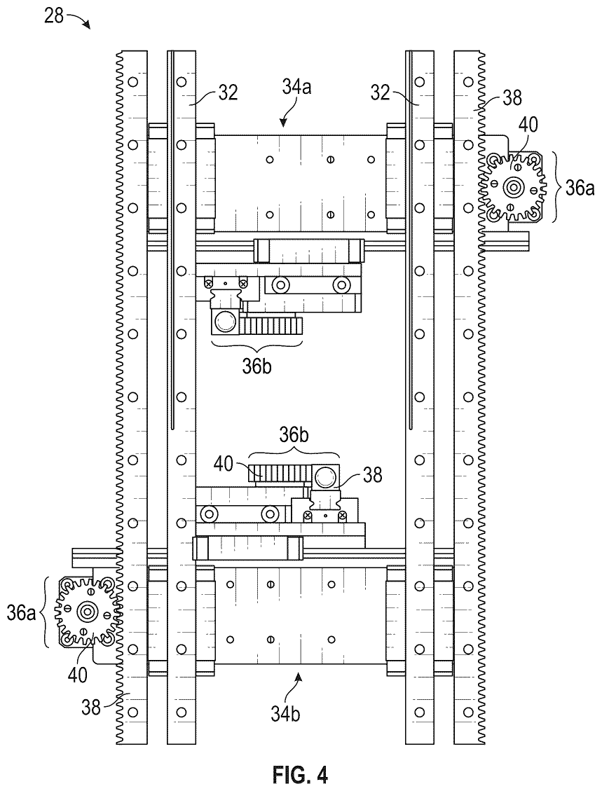

By way of non-limiting illustration, FIG. 3 shows some embodiments of a surgical configuration mechanism 28 including multiple actuation assemblies 34 having actuators 36. By way of further non-limiting illustration, FIG. 4 shows some embodiments of a surgical configuration mechanism having a first actuation assembly 34a and a second actuation assembly 34b, each one comprising a first actuator 36a and a second actuator 36b, where the actuators include rack 38 and pinion 40 mechanisms. Accordingly, the actuation assemblies are configured to move with respect to the frame 32, which in the case of FIG. 4 includes linear rails for easy translation.

According to some embodiments, the actuators 36 are configured to allow a position of a digital representation to be updated when the proxy 26 is physically manipulated (e.g., the actuator includes hardware and software that allows its position to be tracked even when it is moved by an external force, such as a practitioner adjusting the position). For example, some embodiments utilize one or more servomotors or other actuators that include or are otherwise associated with one or more encoders (or other components) configured to track the position. That said, some embodiments include separate encoders (e.g., added proximate to or functionally coupled to the actuators) to digitally update the position of the proxies.

In some embodiments, the actuators 36 include or are otherwise associated with one or more features or additional components (e.g., sensors) that allow a user to measure applied forces. For example, some embodiments use servomotors with built-in force-sensing capabilities. As a result, in some embodiments, one or more proxies 26 can be held in place to prevent accidental manipulation, but they can still be moved or driven into place when a high enough force is applied.

In some embodiments, one or more of the actuators 36 are configured simulate one or more stiffnesses (e.g., of the spine, of the operative level, of one or more components, or otherwise). For example, in some cases, one or more of the actuators are configured to move when an external force is applied to the associated proxies 26 (e.g., via manual manipulation or otherwise), but the movement is configured to match (or substantially match; e.g., within 20% of) the movement that would be experienced in vivo (because the actuators cause the stiffness of the configuration of the proxies to simulate the stiffness of the spine or the applicable portion thereof (or the components coupled thereto). Thus, in some cases, the surgical configuration model 28 allows for a practitioner to evaluate one or more forces applied during a procedure without (or before) applying them directly to the spine (e.g., by applying them to the surgical configuration model).

Where one or more of the actuators 36 are configured to simulate a stiffness, this can be done in any suitable manner. For example, the actuators can be configured to receive stiffness values to simulate as input, which values can be obtained through any suitable means. In some embodiments, the values are: pre-set or pre-provided (e.g., with values corresponding to theoretical stiffness values of various levels of the spine); provided by a practitioner; measured (e.g., using one or more instruments, which (in some cases) can be configured to automatically provide the input values to the actuators, or which can simply obtain the measurements that can be separately uploaded to the surgical configuration model 20; calculated (e.g., based on movements of the proxies 26 or associated components; or otherwise obtained. In some embodiments, different values are provided that correspond with different levels of the spine. In some cases, different values are provided along different axes (e.g., lateral bending may be stiffer, in some cases, than flexion-extension (or vice versa)). In some cases, the simulated stiffness is linear, and in some cases it is non-linear (in some cases, linear and non-linear stiffnesses are both used).

Where simulated stiffnesses are used, the simulated stiffnesses can include any suitable stiffnesses as may be experienced during a surgical procedure. For example, some embodiments are configured to provide between 1 MPa and 10 GPa, or any subrange thereof (e.g., between 10 MPA and 1 GPa, 50 MPa and 800 MPa, 100 MPa and 700 MPa, or any other suitable subrange).

In some embodiments, the surgical configuration model 28 includes one or more locking mechanisms configured to be selectively engaged to prevent one or more proxies 26 from being moved when the locking mechanism is engaged, but allows for the proxies to be manually manipulated when the locking mechanism is disengaged. Where such a locking mechanism is included, the locking mechanism can be incorporated in any suitable manner (e.g., it can include a button, a digital interface, a switch, a mechanical engagement system, or any other suitable mechanism configured to—digitally, mechanically, or otherwise—selectively disable and enable manual movement of the proxies).

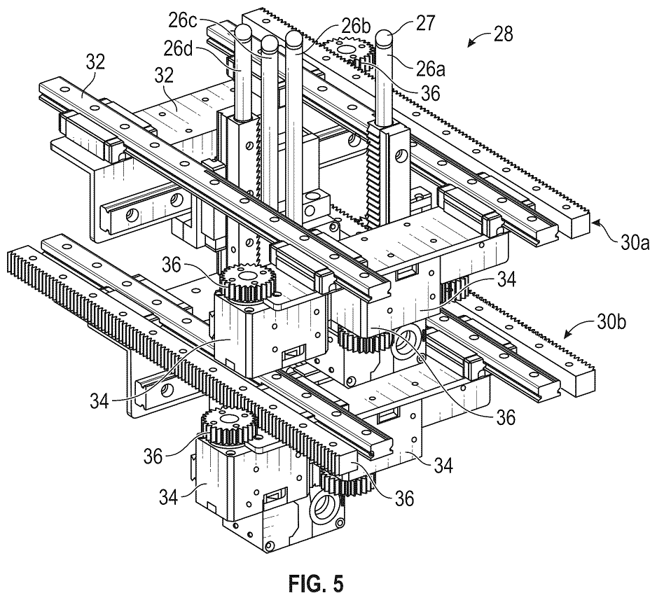

The actuation assembly 34 can be configured to adjust the position (e.g., one or more of the location on the x-axis, the location on the y-axis, the location on the z-axis, the rotation along the x-axis, the rotation along the y-axis, the rotation along the z-axis, the size, the configuration, or the shape) of one or more proxies 26 in any suitable manner. For example, in some cases, a single actuation assembly, or a single actuator 36 of an actuation assembly, is responsible for a single degree of freedom for a single proxy (e.g., a first actuator moves a proxy along a z axis, a second actuator moves a proxy along an x axis, a third actuator moves a proxy along a y axis, etc.). In some cases, one actuation assembly, or one actuator, is responsible for multiple degrees of freedom (e.g., x and y axis adjustments) or for multiple proxies (e.g., a first proxy and a second proxy, whether individually (moving independently of each other) or collectively (moving together)). By way of non-limiting illustration, FIG. 4 shows some embodiments of a first actuation assembly 34a and a second actuation assembly 34, each having a first actuator 36a configured to move the actuation assembly along a first axis and a second actuator 36b configured to move the actuation assembly along a second axis (such that any proxies (not pictured) coupled to such actuation assemblies 34a, 34b would also move). By way of further non-limiting illustration, FIG. 5 shows some embodiments of a surgical configuration mechanism 28 having four proxies 26 (a first proxy 26a, a second proxy 26b, a third proxy 26c, and a fourth proxy 26d), each proxy being coupled to an actuation assembly 34, with each actuation assembly having a plurality of actuators 36 such that the actuation assemblies (and congruently, the proxies) are configured to be selectively moved in three degrees of freedom (e.g., x, y, and z locations).

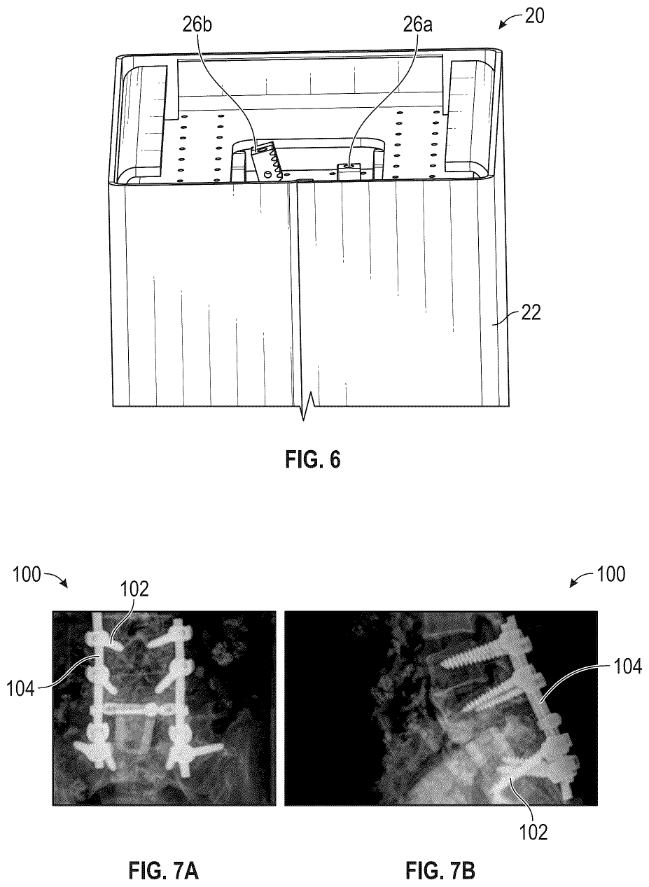

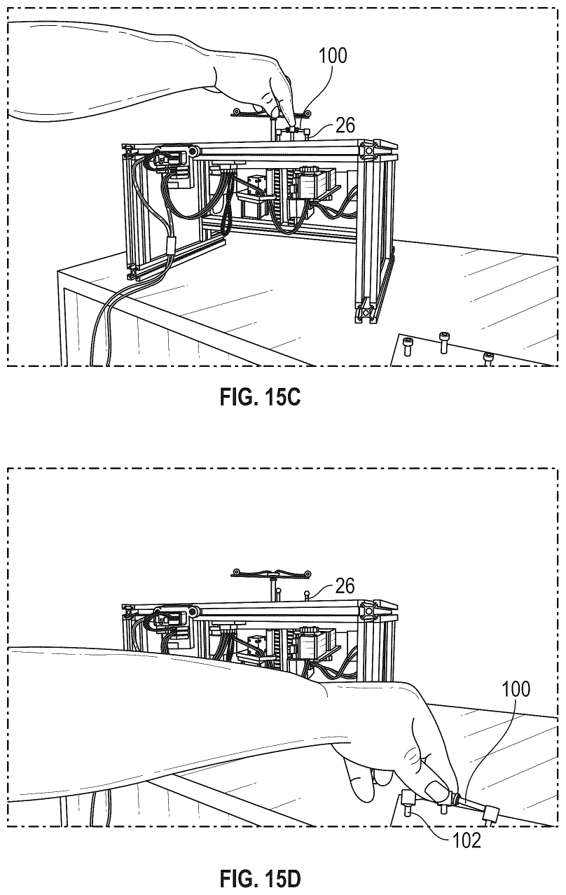

According to some embodiments, the actuation assemblies 34 are configured to rotate one or more proxies 26 (or allow the proxies to be rotated manually). By way of non-limiting illustration, FIG. 6 shows some embodiments of a first proxy 26a with a substantially vertical orientation, and a second proxy 26b that is rotated around the y axis (e.g., the proxy 26b has been biased to account for non-parallel features, such as non-planar screw insertion). Such a feature may be particularly useful where one or more components are configured to be inserted at an angle with respect to one or more other components. For example, FIGS. 7A-7B show x-ray images of a patient's spine with implants 100 implanted therein, the implants 100 including anchors 102 configured to be inserted at various angles with respect to each other, with rods 104 joining the anchors 102 together. Thus, adding one or more additional degrees of freedom may be helpful for simulating off-axis anchor position or limitations in an implant. That said, it is worth mentioning that, in some embodiments, the orientation of the proxies does not necessarily need to match the orientation of the actual components (e.g., anchors), as a mating component (e.g., a rod) can often be rotated after being removed from the proxies (see FIGS. 15C-15D).

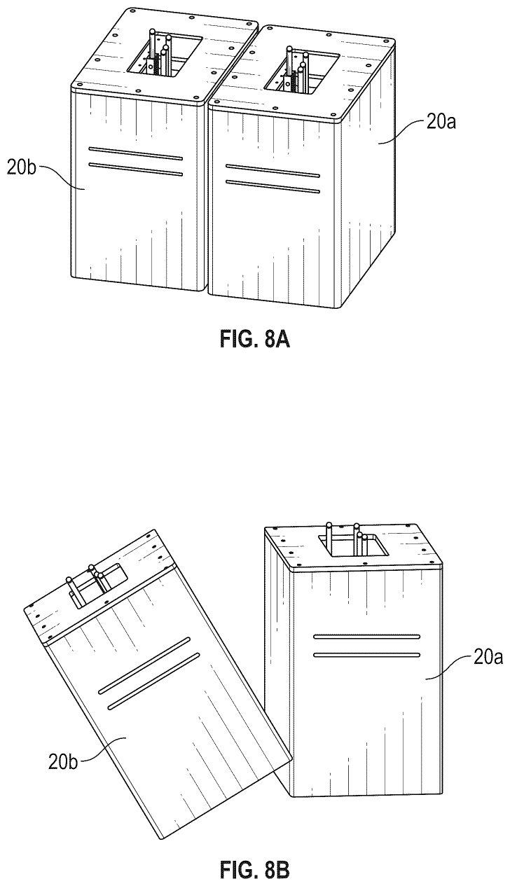

According to some embodiments, multiple surgical configuration models 20 are configured to be used with each other. For example, FIGS. 8A and 8B each show a first surgical configuration model 20a and a second surgical configuration model 20b. Where multiple surgical configuration models are used, any number of them can be used (e.g., 2, 3, 4, 5, 6, 7, or more). In some cases, the surgical configuration models are used at (or are configured to be selectively moved to) an angle with respect to each other (e.g., to simulate anatomical planes), which may be useful in cases such as the one described above in connection with FIGS. 7A-7B. In some cases, multiple surgical configuration models are used to simulate left and right sides of a spine (or another anatomical structure), respectively.

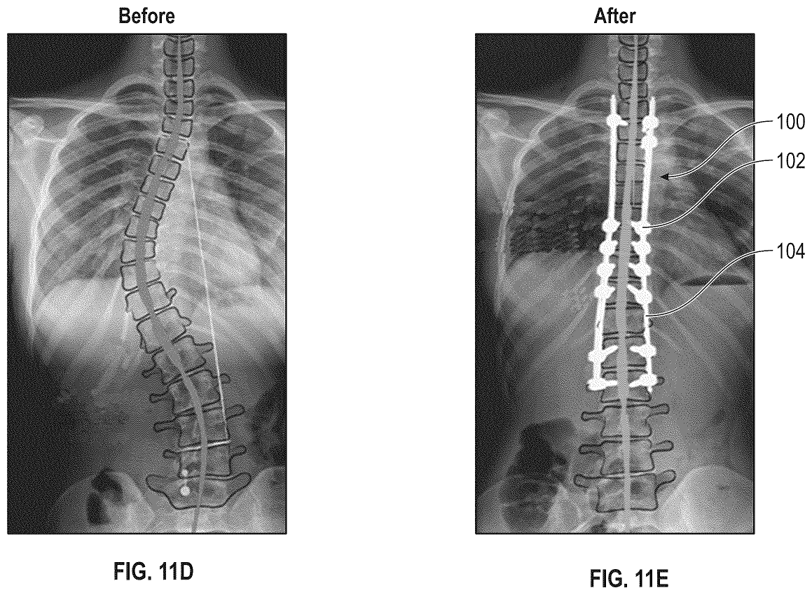

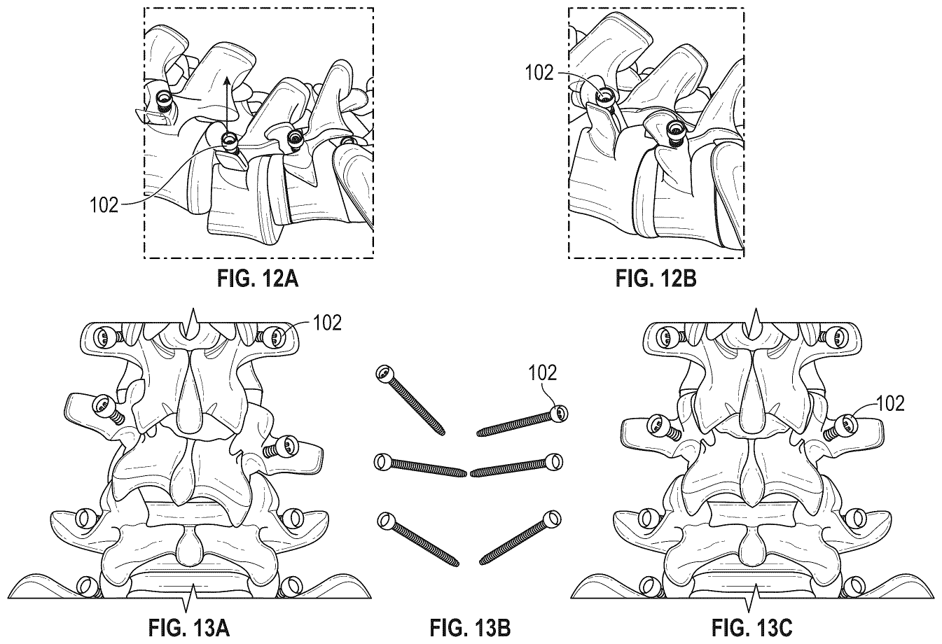

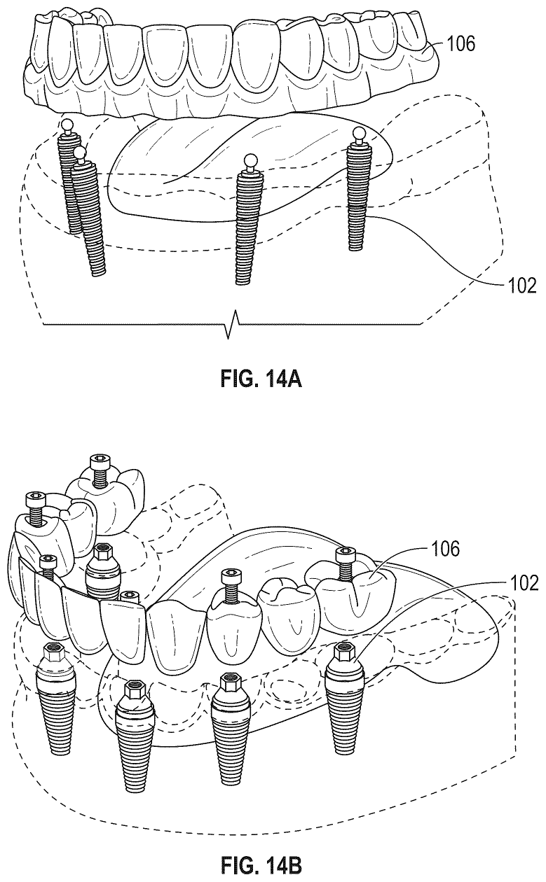

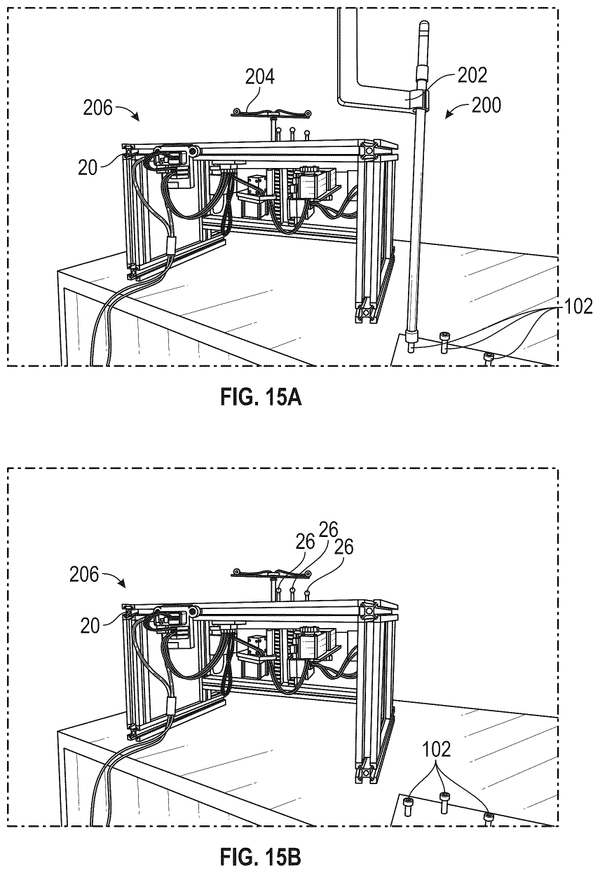

Although there are many procedures—both surgical and non-surgical—in which component tracking in accordance with embodiments of the systems and methods described herein may be useful, some examples include implant installation in connection with spinal surgeries, dental procedures, or other medical applications. For example, in many procedures, surgical anchors (e.g., screws, hooks, wires, nails, or other anchors) are placed into anatomical structures to which subsequent surgical instruments or implants are attached. Examples include: spinal fusion systems, rod constructs, and plates; dental implants; bone fracture reducing pins, screws, and plates; and other sundry orthopedic devices. By way of non-limiting illustration, FIGS. 11A-11C illustrate some embodiments of installation of an implant 100 in connection with a spinal surgical procedure. In such procedure, one or more anchors 102 are installed in spinal vertebrae, and then a rod 104 is affixed to the anchors 102. For assistance with this procedure, a practitioner can use a surgical configuration model 20 (as described herein) to simulate the positions of the anchors 102 with the proxies 26. The practitioner can then form the rod 104 to have the proper configuration using the proxies prior to installing the rod on the actual anchors in the patient (with the rod shaped outside the surgical site, subcutaneous rod passage and coupling can be eased). Accordingly, improper spinal curvature can be more easily remedied (as shown in FIG. 11D, illustrating S-shaped incurvature of the spine, and FIG. 11E, illustrating realignment of the back by arthrodesis rods) using the surgical configuration model. By way of further non-limiting illustration, FIGS. 12A-12B and 13A-13C illustrate some embodiments of additional spinal adjustments that can be more easily performed using a surgical configuration model as a reference for pre-procedure and optimal positions of anchors 102. Similarly, FIGS. 14A-14B show some embodiments of anchors 102 used in connection with dental implants 106, illustrating yet another potential use of a surgical configuration model to simulate implant components prior to and during surgery (e.g., allowing a dental implant to be formed to fit the anchors in the patient's jaw by fitting the simulated anchors on the model).

According to some embodiments, the positions of one or more components (e.g., anchors 102 implanted in a patient's bone during a surgical procedure) is measured and communicated to the surgical configuration model 20 so that the surgical configuration model can accurately simulate such positions (e.g., by placing proxies in appropriate positions to mimic the position of the anchors or other components). Although the components'actual positions can be measured in any suitable manner, some embodiments utilize a stereotactic localization system 200. Where a stereotactic localization system is used, the stereotactic localization system can include any component useful for sensing, measuring, digitizing, calculating, or otherwise processing one or more components. In some cases, the stereotactic localization system includes one or more components as taught in U.S. patent application Ser. No. 18/917,912, entitled SYSTEMS AND METHODS FOR IMPROVED STEREOTACTIC LOCALIZATION, filed Oct. 16, 2024 (Attorney Docket No. 23845.194), the contents of which are incorporated herein by reference in their entirety. Thus, in some embodiments, a stereotactic localization system can identify positions of one or more components in a patient's body, the described model 20 can be used to move proxies to match or otherwise be compatible with the positions of the components in the patient's body, an implant construct can be configured or shaped by being coupled to the proxies so as to be tailored to the patient's particular needs, and the implant construct can be implanted into the patient.

By way of non-limiting illustration, FIG. 15A illustrates that, in accordance with some embodiments, a stereotactic localization system 200 can be used to determine the positions of anchors 102 (e.g., anchors within a patient's body) or other components (for example, by coupling one or more markers 202 to the anchors and sensing them with one or more sensors 204). In some cases, a digital representation 206 of the position of the components is constructed (as shown in FIG. 15A). As shown in FIG. 15B, some embodiments of the surgical configuration model 20 can mimic the positions of the anchors 102 (or other components) using the proxies 26. In some embodiments, one or more processors perform additional calculations (e.g., an ideal configuration for a rod or another additional component, as shown by the digital representation 206 of FIG. 15B). As shown in FIG. 15C, the positions of the proxies 26 can be used to form or correctly tailor an implant 100 (e.g., bend, assemble, or otherwise form a rod or implant such that it will conform to the actual positions of the anchors). As shown in FIG. 15D, the implant 100 can then be inserted into the patient's body, conforming to the positions of the actual anchors 102 just as it did to the proxies 26.

In some embodiments, one or more processors or pieces of software is included (e.g., as set forth in the above incorporated patent application). In some cases, the processor or software is configured to do one or more of the following: display component or proxy 26 position (e.g., display a discrepancy between proxy position and actual anchor position, display proxy position, display anchor position, or otherwise display current or ideal component position data); calculate a suggested shape or configuration of an additional implant component (e.g., calculate the ideal shape of a rod based on anchor positions); display a suggested shape or configuration; update data (e.g., relating to positions) in response to anchor placement, physical manipulation of proxies, algorithmic manipulation of proxies, or otherwise; place one or more proxies into positions corresponding to actual component positions; calculate discrepancies between simulated and actual positions; calculate discrepancies between suggested component shape or configuration and actual component shape or configuration; or display or otherwise output results or data relating to any of the foregoing to a user.

As one example of a way the software may be used, if a first screw and a second screw are each attached to the same vertebral body, the first screw naturally maintains a set distance from the second screw (as they are both coupled to the same rigid body). Accordingly, if the first screw is manipulated, some embodiments of the software are configured to automatically update the position of the second screw (and the corresponding proxy 26) accordingly. In some embodiments, the software is configured to manipulate proxy positions to achieve the most distributed curvature or achieve one or more other objectives (e.g., a solution to correct a physiological abnormality).

According to some embodiments, one or more portions of the surgical configuration model 20 are kept in a sterile environment to ensure that components used in connection with the surgical configuration model are not contaminated. This can be done in any suitable manner. In some embodiments, the surgical configuration model is formed of materials configured to be autoclaved (e.g., metal or metal alloys). In some embodiments, the surgical configuration model or portions thereof (e.g., proxies 26) include sterile coverings, shrouds, or bags (similar to bags for covering a C-arm, or other sterile instrument bags) that are disposable and can be used to cover the surgical configuration model or its constituent components. In some embodiments, one or more proxies (or proxy heads 27) include built-in disposable bags, or the proxies (or proxy heads) themselves are disposable.

According to some embodiments, one or more methods for modeling one or more aspects of a surgical procedure are provided. The methods can include obtaining (e.g., forming, assembling, modifying existing materials to produce, manufacturing, purchasing, or otherwise obtaining) any of the components described herein. For example, some embodiments include obtaining one or more surgical configuration models 20, implants 100, stereotactic localization systems 200, or any parts of any of the foregoing.

The methods can also include using any of the components described herein in any suitable manner (e.g., in accordance with any of the functionality described herein). For example, wherever a component is configured to perform a particular function, the method can include performing that function, performing that function using that component, causing the component to perform the function, or taking additional actions in connection with the component's performance of that function. Some illustrative examples of portions of the method are included below.

According to some embodiments, the described methods include installing a first surgical component (e.g., an anchor 102, a nail, a rod 104, a mesh, an implant, or any other suitable surgical component) into a patient. That said, in some cases, a first surgical component is used that is already present inside a patient's body (e.g., an implant installed in a previous surgery, a vertebra or other bone, or another already present component).

In some embodiments, the methods include installing a second surgical component (e.g., any suitable type of surgical component as discussed herein, whether the same type as the first surgical component or a different type). By way of non-limiting example, some embodiments of the first surgical component and the second surgical component are each pedicle screws or other anchors 102 for use in a spinal fusion surgery. Some embodiments include installing additional surgical components (of any suitable variety, in any suitable number).

Some embodiments of the methods include measuring (using a stereotactic localization system 200, a sensor, manual measurements, calipers, protractors, rulers, or otherwise) the positions of the surgical components (with respect to each other, with respect to one or more portions of the patient, or otherwise). For example, some embodiments include measuring a position (e.g., measuring one or more positions or one or more components, such as an orientation, an x-position, a y-position, a z-position, or others) of an anchor 102 with respect to a vertebra in which it is installed, with respect to another anchor, or with respect to any other suitable reference point.

Some embodiments of the methods include using a surgical configuration model 20 to simulate one or more aspects of the surgical procedure, such as by using one or more proxies 26 to simulate the positions of one or more corresponding components. Indeed, in some embodiments, a surgical configuration mechanism 28 of the surgical configuration model adjusts the proxies to match the measurements of the components. Thus, in some cases, the methods result in the creation of a template or external model that replicates (or is specifically tailored to) an interior condition of the patient.

In some cases, the methods include using the external model to create, form, modify, adjust, evaluate, measure, or otherwise affect or assess one or more additional components. For example, some embodiments include bending a rod 104 (or otherwise modifying a component) to fit with the proxies 26 so that the rod (or other component) will fit with the anchors 102 (or other interior components) without the need to make the requisite modifications or adjustments inside the surgical wound.

Some embodiments include evaluating the proxies in order to ascertain information about the installed components. For example, some embodiments include testing to see how much force would be required to displace one or more components a certain distance based on how much force is required to displace the proxies. Some embodiments include manually (or otherwise) repositioning the proxies and evaluating what would need to be done in order to carry out a similar effect for the components that the proxies represent.

According to some embodiments, the methods include outputting information (e.g., via a display, loudspeaker, or any other output device). The information can include any suitable information, such as any information measured, calculated, or simulated regarding the surgical components, the proxies 26, other portions of the surgical configuration model 20, any implants 100 used, the stereotactic localization system 200, or any other suitable aspect of the procedure. For example, some embodiments are configured to output measurements (of components, proxies, distances between components or proxies, displacements of components or proxies, forces, or other measurements), suggestions (e.g., in the form of instructions, illustrations, suggested measurements, suggested modifications, suggested dimensions, or otherwise), calculations, additional information regarding the surgical procedure (e.g., time elapsed, patient vitals, patient charts, or other such information), or any other suitable output.

According to some embodiments, the methods include performing one or more additional portions of the surgical procedure using one or more components that have been created, modified, or evaluated with the assistance of the surgical configuration model. For example, some embodiments include installation of an implant (or part thereof) that was formed to fit with the proxies 26. Some embodiments include modification of an implant (or part thereof) based on measurements or other information obtained from the proxies. Thus, some embodiments of the surgical configuration model 20 can be used in many extremely useful ways during a surgical procedure once the model has been properly configured (to simulate corollary components used in the surgical procedure).

The described systems and methods can be modified in any suitable manner. For instance, some embodiments of the surgical configuration model are integrated or otherwise used with the stereotactic localization system (e.g., by having markers formed thereon, by being integrated with the processor, or otherwise).

As another example of a possible modification, some embodiments are configured to be used remotely. For instance, anchors 102 (or other components) may be inserted, the position of the anchors (or other components) ascertained and transmitted to a lab or other remote location having the surgical configuration model. The lab (or other location) can then use the surgical configuration model to simulate the positions of the anchors (or other components) to form a fitting implant (e.g., by casting, pressing, 3D printing, or otherwise forming attachment points for the anchors (or other components)) remotely. The implant can then be attached at a later surgery date, or it can transported to (or formed at (e.g., via additive manufacturing)) the surgical location during the procedure (especially if the remote location is near the operation location).

As another example of a suitable modification, the stereotactic localization system 200 of some embodiments is used to measure one or more characteristics of the proxies 26. In some cases, such a technique helps to ensure that the proxies better match the configuration of the surgical components they are configured to simulate. In some cases, such a technique is used to calibrate the proxies, the software, the stereotactic localization system, or any other parts of the overall system.

In addition to the aforementioned features, the described systems and methods can include any other suitable feature. Indeed, some embodiments allow practitioners to experiment with surgical equipment (i.e., implants) in a safe, sterile, and representative environment before transferring components to an actual patient where experimentation may be riskier. Moreover, embodiments of the described systems and methods can allow for the following: locating the 3D position of one or more anchors 102 or other components inter-op (during an operation); replicating the 3D position of the anchors or other components in an ex-vivo surrogate; virtually recreating and displaying the proxies or other proxy components; virtually relating bones and other patient anatomy to the anchors or other components; manipulating the position of the anchors or other components outside the surgical site through direct physical manipulation, indirect computer manipulation, or otherwise; updating a computer rendering of the anchors or other components, which may also include updating the position of one or more anatomical structures attached to the anchors or other components (in 2D or 3D); forming or shaping an implant based on the proxy anchors or other components; and attaching the implant to the in-situ anchors or other components.

In situations where multiple anchors are attached to a single bone, different manipulations are possible. For example, spondylolisthesis can, in some cases, be reduced by causing a pair of anchors 102 to co-translate (e.g., when one anchor is retracted, a second anchor can be assigned to translate with it). An example of this can be seen in FIGS. 12A-12B. As another example, in scoliosis manipulation, one anchor can be pushed up, while the other anchor is pulled down. Additional degrees of freedom can be calculated and rotation applied as appropriate. An example of this can be seen in FIGS. 13A-13C.

Any and all of the components in the figures, embodiments, implementations, instances, models, cases, methods, applications, iterations, elements, and other parts of this disclosure can be combined with each other in any suitable manner. Additionally, any component of any system and method described herein can be removed, separated from other components, modified with or without modification of like components, substituted, omitted, replaced, performed in series, performed in parallel, made to overlap, made not to overlap, or can otherwise be altered together or separately from anything else disclosed herein.

As used herein, the singular forms “a”, “an”, “the” and other singular references include plural referents, and plural references include the singular, unless the context clearly dictates otherwise. For example, reference to a fastener includes reference to one or more fasteners, and reference to actuators includes reference to one or more actuators. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. A surgical configuration model comprising:
a first proxy configured to represent a first surgical component; and
a surgical configuration mechanism configured to cause the first proxy to simulate a position of the first surgical component during a surgical procedure.

2. The surgical configuration model of claim 1, further comprising a second proxy configured to represent a second surgical component, wherein the surgical configuration mechanism is configured to move to cause the second proxy to simulate a position of the second surgical component during the surgical procedure.

3. The surgical configuration model of claim 1, wherein the first proxy is coupled to an actuator configured to adjust the position of the first proxy.

4. The surgical configuration model of claim 2, wherein the first proxy is coupled to an actuator configured to adjust the position of the first proxy with respect to the second proxy.

5. The surgical configuration model of claim 1, wherein the first proxy is selectively interchangeable with another proxy.

6. The surgical configuration model of claim 1, wherein the first proxy comprises a first proxy head that is selectively interchangeable with another proxy head.

7. The surgical configuration model of claim 1, wherein the first proxy is configured to simulate an orthopedic anchor.

8. The surgical configuration model of claim 1, further comprising a housing, wherein the first proxy is configured to extend from an opening in the housing.

9. A system comprising:
a sensor configured to sense a position of a first component during a surgical procedure; and
a spinal configuration model comprising:
a first proxy configured to represent the first component; and
a surgical configuration mechanism configured to cause the first proxy to move to simulate the position of the first component.

10. The system of claim 9, wherein the sensor is further configured to sense a position of a second component with respect to the position of the first component during the surgical procedure, wherein the spinal configuration model further comprises a second proxy configured to represent the second component, and wherein the surgical configuration mechanism is further configured to cause the second proxy to move to simulate the position of the second component with respect to the position of the first component.

11. The system of claim 9, wherein the position of the first component comprises at least one of an x-position, a y-position, a z-position.

12. The system of claim 9, further comprising software configured to communicate with the surgical configuration mechanism.

13. The system of claim 9, wherein the software is configured to calculate a desirable position of the first component and to cause the surgical configuration mechanism to place the first proxy in a position simulating the desirable position of the first component.

14. The system of claim 9, wherein the sensor comprises a stereotactic camera array having a first camera and a second camera.

15. A method for modeling an aspect of a surgical procedure, the method comprising:
obtaining a surgical configuration model comprising:
a first proxy configured to represent a first surgical component; and
a surgical configuration mechanism configured to cause the first proxy to simulate a position of the first surgical component during the surgical procedure; and
causing the first proxy to simulate the position of the first surgical component.

16. The method of claim 15, wherein the surgical configuration model further comprises a second proxy configured to represent a second surgical component, and wherein the method further comprises causing the second proxy to simulate a position of the second surgical component with respect to the position of the first surgical component.

17. The method of claim 15, wherein the causing the first proxy to simulate the position of the first surgical component comprises causing the first proxy to simulate at least one of an x-location, a y-location, a z-location, and an orientation of the first proxy.

18. The method of claim 15, further comprising:
obtaining a stereotactic localization system;
using the stereotactic localization system to detect the position of the first surgical component; and
using software to cause the surgical configuration mechanism to adjust the first proxy in accordance with data obtained from the stereotactic localization system.

19. The method of claim 15, wherein the surgical configuration mechanism is configured to detect a change in a position of the first proxy, and wherein the method further comprises:
manually adjusting the position of the first proxy; and
detecting the change in the position of the first proxy using the surgical configuration mechanism.

20. The method of claim 15, further comprising configuring a second surgical component using the first proxy as a guide and installing the second surgical component proximate the first surgical component.
